(19) United States
(12) Patent Application Publication
(10) Pub. No.: US 2011/0003382 A1
(43) Pub. Date: Jan. 6, 2011

(54) USE OF CORTICOSTERONE FOR INCREASING EGG ANTIBODY

(75) Inventors: Mark Cook, Madison, WI (US); David Trott, Guelph (CA)

Correspondence Address: Patent Docket Department;Armstrong Teasdale LLP, 7700 Forsyth Boulevard, Suite 1800, St. Louis, MO, 63105, US

(73) Assignee: WISCONSIN ALUMNI RESEARCH FOUNDATION, Madison, WI (US)

(21) Appl. No.: 12/496,223

(22) Filed: Jul. 1, 2009

(51) Int. Cl. C12N 5/06; A61K 39/00

(52) U.S. Cl. 435/326; 424/184.1

(57) ABSTRACT

Methods for increasing antigen-specific egg yolk antibody titer and eggs having increased antigen-specific egg yolk antibody titer are disclosed. More specifically, the method for increasing antibody titer may include the steps of: administering to an egg-laying animal a corticosteroid; and then, exposing the animal to an immunogenic dose of the antigen.

### BACKGROUND OF THE DISCLOSURE

The present disclosure relates generally to eggs having an increased antibody titer and to methods for making the same, and more particularly to methods for increasing egg yolk antibody titer.

In addition to the protective properties conferred by antibodies in vivo in the humoral immune system, antigen-specific antibodies have commercial value, for example, for use as (1) an animal feed supplement, (2) a diagnostic reagent for use in clinical and research laboratory settings and (3) active and passive vaccines. Antibody-containing feed supplements can prevent and can treat infectious disease, can promote growth, can improve feed conversion and can increase yield of animal products such as meat, milk and eggs. Antibodies are advantageous prophylactic and therapeutic alternatives to antibiotics in feed supplements because they do not promote resistance of animal and human pathogens to anti-pathogen drugs, because they do not accumulate in the animal products, and because they are less expensive to develop and produce.

Antibodies produced in egg-laying animals, specifically IgY antibodies, find particular utility in immunological assays. Such antibodies do not (1) cross-react with mammalian IgG, (2) bind to Fc receptors, (3) interact with rheumatoid factors or (4) react with HAMA (human anti-murine antibodies), so non-specific binding is low. Also, secondary antibody-enzyme conjugates made with egg yolk antibodies need not be adsorbed with a mammalian protein to reduce background, as is required for most conjugates that employ mammalian secondary antibody. Conventional chicken egg yolks contain approximately 100-150 mg of IgY immunoglobulin. Unlike yolk, egg albumin contains much lower concentrations of IgY. Each doubling of the antibody titer in an egg reduces the cost of producing an antibody product by 50%.

Methods for producing antibodies, including human monoclonal antibodies, in an egg-laying animal are known to those of skill in the art. Such methods generally include the step of immunizing the animal with an antigen, whereupon serum antibodies to the antigen are accumulated by transporters in the eggs, and particularly in the egg yolks. See Bar-Joseph M & Malkinson M, “Hen egg yolk as a source of antiviral antibodies in the enzyme-linked immunosorbent assay (ELISA): a comparison of two plant viruses,” J. Virol. Methods 1:179-183 (1980); Gassmann M, et al., “Efficient production of chicken egg yolk antibodies against a conserved mammalian protein,” FASEB J. 4:2528-2532 (1990); and Zhu L, et al., “Production of human monoclonal antibody in eggs of chimeric chickens,” Nature Biotechnology 23:1159-1169 (2005), each of which is incorporated herein by reference as if set forth in its entirety. Furthermore, the passive transfer of antibody from egg-laying animals such as hens to the egg yolk has recently been used as a commercial means for mass production of polyclonal antibodies. See Cook, M. E., 2004, “Antibodies: Alternatives to antibiotics in improving growth and feed efficiency,” J. Appl. Poult. Res. 13:106-119; Kovacs-Nolan & Mine, 2004, “Avian egg antibodies: basic and potential applications,” Avian Poultry Biol. Rev. 15:25-46; and Schade, et al., 2005, “Chicken egg yolk antibodies (IgY-technology): A review of progress in production and use in research and human and veterinary medicine,” ATLA, 33(2):129-154. It would be beneficial, however, to provide methods of increasing the concentration or total quantity of antigen-specific antibody in the egg yolk. These methods would benefit both the producer of polyclonal antibody and the producer of hatching eggs.

Accordingly, there is a need in the art for increasing antibody titer in eggs, particularly in egg yolks to further reduce the cost of egg-derived antibodies. It would be further advantageous if the methods for increasing antibody titer could also show an increased antibody response in the avian animal to soluble protein antigens.

### BRIEF DESCRIPTION OF THE DISCLOSURE

Accordingly, the present disclosure is generally directed to a method for increasing antigen-specific egg yolk antibody titer. More specifically, in one aspect, the method for increasing antibody titer includes the steps of: administering to an egg-laying animal a corticosteroid; and then, exposing the animal to an immunogenic dose of the antigen. The animal can be exposed to the antigen at least about thirty minutes, at least about twenty four hours after administration of the corticosteroid, or at least about forty-eight hours after administration of the corticosteroid.

In some embodiments, the egg is an avian egg and can be a chicken, quail, pheasant, duck, emu, goose, ostrich or turkey egg.

In another aspect, the present disclosure is directed to an egg laid by an egg-laying animal treated with an immunogenic dose of an antigen of interest after exposure to a corticosteroid. Particularly, the egg has an antigen-specific antibody titer at least 2.0-fold higher than that in an egg laid by an animal exposed to an immunogenic dose of the antigen without prior exposure to the corticosteroid.

In some embodiments, the egg-laying animal is an avian animal and can be a chicken, quail, pheasant, duck, emu, goose, ostrich or turkey.

In some embodiments, the corticosteroid is corticosterone, dexamethasone, or cortisol.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 shows the effect over time of feed restriction on phospholipase A₂ (PLA₂) antibody titer in egg yolk.

FIG. 2A shows the effect over time of orally administered corticosterone on peripherial blood mononuclear cell (PBMC) count.

FIG. 2B shows the effect over time of orally administered corticosterone on body weight (BW) change.

FIG. 2C shows the effect over time of orally administered corticosterone on egg production.

FIG. 3A shows the effect over time of orally administered corticosterone on PLA₂ antibody titer in serum.

FIG. 3B shows the effect over time of orally administered corticosterone on PLA₂ antibody titer in egg yolk.

FIG. 4A shows the effect over time of orally administered corticosterone on peripheral peripherial blood mononuclear cell (PBMC) count.

FIG. 4B shows the effect over time of orally administered corticosterone on body weight (BW) change.

FIG. 4C shows the effect over time of orally administered corticosterone on egg production or laying frequency.

FIG. 5 shows the effect over time of orally administered corticosterone on egg yolk antibody titer.

FIG. 6 shows the effect over time of orally administered corticosterone on egg production.

FIG. 7 shows the biosynthetic pathway involving corticosteroids such as corticosterone and cortisol.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF PREFERRED EMBODIMENTS

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The augmentation of humoral antibody response after stress-induced activation of the hypothalamic-pituitary-adrenal (HPA) axis is an established neuroendrocrine-immune interaction reported in mammals (Dhabhar, 2002, “Stress-induced augmentation of immune function—the role of hormones, leukocyte trafficking, and cytokines,” Brain Behavior and Immunity, 16:785-798). Corticosterone (CORT) released by the adrenal gland is the end product of stress-induced activation of HPA-axis. In mice, acute stress and exogenous corticosterone increased mouse T-dependent antibody response to sheep erythrocytes (Stanulis et al., 1996, “Role of corticosterone in the enhancement of the antibody response after acute cocaine administration,” J. Pharm. Exp. Therapeutics, 280:284-291). Neuroendocrine-immune interactions have also been studied extensively in avian species; however, few studies have been conducted with the objective of increasing antibody response to soluble protein antigen by exposing birds to acute stress or exogenous corticosterone.

Typically, stress and plasma corticosterone (CORT) levels are inversely associated with avian antibody response to immunization, however, it has been found in the present disclosure that short-term administration of dietary CORT during immunization of laying hens would increase hen antibody response to soluble protein antigen (SPA).

As used herein, a corticosteroid is defined as any of the steroid hormones made by the cortex (outer layer) of the adrenal gland. More particularly, corticosteroids act on the immune system by blocking the production of substances that trigger allergic and inflammatory actions, such as prostaglandins. Corticosteroids are involved in a wide range of physiologic systems such as stress response, immune response and regulation of inflammation, carbohydrate metabolism, protein catabolism, blood electrolyte levels, and behavior.

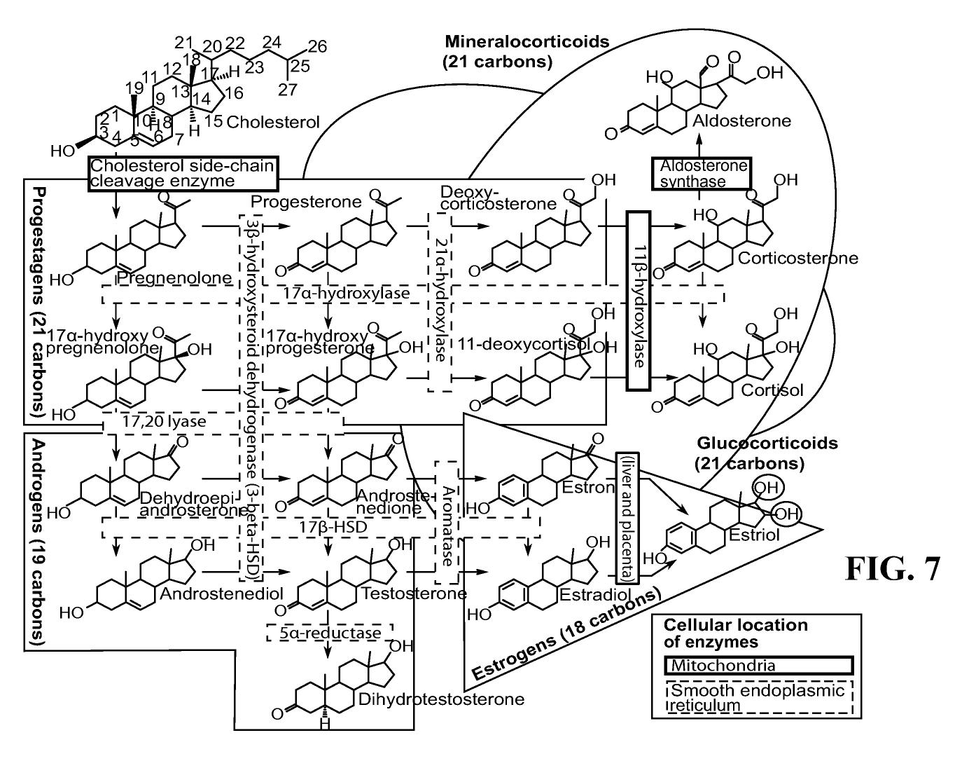

In some embodiments, the natural corticosteroids for use in the methods of the present disclosure suitably include corticosterone, dexamethasone, and cortisol. In one particularly preferred embodiment, the corticosteroid is the primary natural hormone found in avians, corticosterone. Aldosterone and corticosterone share the first part of the biosynthetic pathway for corticosteroids (see FIG. 7). The last part is either mediated by the aldosterone synthase (for aldosterone) or by the 11β-hydroxylase (for corticosterone). These enzymes are nearly identical (they share 11β-hydroxylation and 18-hydroxylation functions), but aldosterone synthase is also able to perform an 18-oxidation. Moreover, aldosterone synthase is found within the zona glomerulosa at the outer edge of the adrenal cortex; 11β-hydroxylase is found in the zona fasciculata and reticularis. It should be noted that while natural corticosteroids are described herein, the methods may use synthetic corticosteroids without departing from the scope of the present disclosure.

In some embodiments of the methods of the present disclosure, the corticosteroid is administered to the egg-laying animal prior to exposing the animal to the target antigen. Any suitable methods as known in the art for administering the corticosteroid may be used without departing from the scope of the present disclosure. For example, the corticosteroid may be administered to the egg-laying animal orally, parenterally, intraperitoneally, intravenously, intradermally, or intrathecally. In one embodiment, the corticosteriod can be used with capsules, tablets, pills, powders, and granules to be added to feed for oral administration. In such solid dosage forms, the corticosteriod is ordinarily combined with one or more adjuvants appropriate to the indicated route of administration. If administered in capsules or tablets, the corticosteriod can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and or polyvinyl alcohol, and then tableted or encapsulated for convenient administration. Such capsules or tablets can contain a controlled-release formulation such as can be provided in a dispersion of the corticosteriod in hydroxypropylmethyl cellulose. In the case of capsules, tablets, and pills, the corticosteriod can also comprise buffering agents such as sodium citrate, or magnesium or calcium carbonate or bicarbonate. Tablets and pills can additionally be prepared with enteric coatings. In another embodiment, the corticosteroid is delivered using an implanted time released delivery system made of blends of material such as caprolactone, polyglycolic acid, and polylactic acid.

Liquid dosage forms for oral administration can include pharmaceutically acceptable emulsions, solutions, suspensions, syrups, and elixirs containing inert diluents commonly used in the art, such as water. Such delivery systems can also comprise adjuvants, such as wetting agents, emulsifying and suspending agents, and sweetening, flavoring, and perfuming agents, in addition to the target corticosteroid. In one particular embodiment, the corticosteroid is administered in water. As corticosteroids are typically insoluble in water, the corticosteroid of this embodiment must first be dissolved in ethanol and then added to water for administration to the egg-laying animal.

In another embodiment, the drug can be injected into the egg-laying animal. Depending upon the carrier component used, the corticosteriod can be contacted with the animal parenterally, intraperitoneally, intratumor, or intrapleural. The term “parental” as used herein includes subcutaneous, intravenous, intramuscular, or intrasternal injection, or infusion/implantation technique.

Injectable drug preparations, for example sterile injectable aqueous or oleaginous suspensions, can be formulated according to the known art using suitable dispersing or wetting agents and suspending agents. Among the acceptable vehicles that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can also be used. Mixtures of solvents and wetting agents discussed herein are also useful.

As used herein, an amount of corticosteriod is sufficient if it increases the antigen-specific egg yolk antibody titer by at least about 2.0-fold, more suitably, at least about 2.5-fold, more suitably, at least about 3.0-fold, even more suitably, at least about 3.5-fold, and even more suitably, at least about 5.0-fold, relative to the titer obtained in eggs from egg-laying animals exposed to the antigen but not treated with the corticosteriod.

Typically, the corticosteroid can be administered for a period of at least one day. More suitably, the corticosteroid can be administered for a period of at least two days, even more suitably, at least five days, and even more suitably, at least six days. It should be understood that the corticosteroid can be administered once daily for the time periods above, or can be administered more than once daily for the time periods above, such as twice daily, three times daily, four times daily, and so forth, so long as the corticosteroid is administered in such an amount as to increase titer of the antigen-specific antibody relative to the titer of antibody specific to the antigen in an animal exposed to the immunogenic dose without corticosteroid.

In some embodiments, the administered amount of the corticosteroid is between about 1.0 mg and 10 mg, per animal per day. This can be accomplished using a corticosteroid-supplemented animal feed containing from about 10 mg to about 90 mg of corticosteroid per kg of feed, and more suitably about 30 mg of corticosteroid per kg of feed. In an alternative embodiment, a sufficient amount of corticosteroid is administered in water, using from about 5 mg to about 45 mg of corticosteroid per kg water.

The egg-laying animal can be, e.g., an avian animal, a mammal, a marsupial, a reptile or an amphibian. The animal can be exposed to any antigenic agent against which a humoral immune response can be raised in the animal. The antigen can be a pathogenic agent such as a virus, a bacterium, a fungus, a protozoan, or an antigenic epitope of a pathogenic agent or any other agent against which a humoral response can be raised, such as, but not limited to, a cell surface marker, such as a cancer cell marker. Furthermore, the antigen can be a nucleic acid molecule that encodes an antigen or antigenic determinant, as in published U.S. Patent Publication No. 2004/0087522, incorporated herein by reference as if set forth in its entirety.

The animal can be exposed to more than one antigen (or more than one epitope) such that more than one antigen-specific antibody is produced and transported to the egg yolk.

A suitable immunogenic dose of antigen is 50-500 μg/ml for a 1 ml injection of an emulsion containing a purified antigen. Alternatively, a suitable immunogenic dose is 3 mg/ml for a 1 ml injection of an emulsion containing an unpurified antigen.

In some embodiments, the antigen is administered to the egg-laying animal at least thirty minutes after administration of the corticosteroid. More suitably, the antigen can be administered to the animal at least twenty-four hours after administration of the corticosteroid, and even more suitably, the antigen can be administered at least forty-eight hours after administration of the corticosteroid.

In addition to the methods of making an egg having a yolk that comprises an antigen-specific antibody, the present disclosure is directed to the egg made therefrom. Specifically, by using the methods described above, eggs having an antigen-specific egg yolk antibody titer of at least from about 0.28 mg/ml to about 0.5 mg/ml are produced.

Antibodies can be prepared from the egg yolks using conventional methods available to the skilled artisan. Briefly, yolks can be freeze dried to form a shelf-stable powdered egg yolk product. Yolk antibodies can be purified, e.g., to remove large quantities of lipid. See Camenisch C, et al., “General applicability of chicken egg yolk antibodies: the performance of IgY immunoglobulins raised against the hypoxia-inducible factor la,” The FASEB Journal 13:81-88 (1999); Akita E & Nakai S, “Comparison of four purification methods for the production of immunoglobulins from eggs laid by hens immunized with an enterotoxigenic E. coli strain,” J. Immunol. Methods 160:207-214 (1993), each incorporated by reference as if set forth herein in its entirety; as well as incorporated U.S. Pat. Publication No. 2004/0087522. Commercially available egg antibody purification kits, such as EGGstract® IgY Purification Systems (Promega; Madison, Wis.) or Eggcellent® Chicken IgY Purification (Pierce Biotechnology, Inc.; Rockford, Ill.), can also be used to purify the antibodies.

The antibodies themselves can be purified to the required extent and employed in the manner in which antibodies obtained from other sources are used. For example, the antibodies can be used as a feed supplement when mixed with animal feed, or as a passive vaccine when mixed with a pharmaceutically acceptable carrier, or as a diagnostic reagent, especially when provided in a kit with other reagents for a diagnostic assay. Acceptable uses for the antibodies include flow cytometry, Western blotting, immunohistochemistry, latex agglutination and ELISA.

The disclosure will be more fully understood upon consideration of the following non-limiting Examples.

### EXAMPLES

In all Examples, Single Comb White Leghorn hens (SCWL) used were transported as pullets (18- to 22-weeks of age) from S&R Farms (Whitewater, Wis.) to University of Wisconsin—Madison Poultry Research Laboratory. Hens were individually housed in cages with raised bottom floors for at least four weeks before being assigned to an Example, and were 26- to 60-weeks of age at the beginning of experiments. Hens were fed, ad libitum, a standard corn-soybean meal based laying hen diet formulated to meet nutritional requirements. Housing was maintained under an automated lighting schedule with 16:8-h light-dark cycle. All procedures involving animals were approved by Animal Care Committee at University of Wisconsin, Madison.

The antigen selected for comparing the antibody response of hens was a 13 kDa soluble protein antigen, phospholipase A₂ (PLA₂) purified from porcine pancreas (Novozyme, Bagsvaerd, Denmark; greater than 90% purity based on SDS-PAGE). PLA₂ was used because of its commercial relevance, and its previous use as a model antigen for studying hen antibody response (Trott, et al., 2008, “Additions of killed whole cell bacteria preparations to Freund complete adjuvant alter laying hen antibody response to soluble protein antigen,” Poult. Sci., 87(5):912-917). Hens were immunized according to methods described by Trott, et al. (2008). The primary immunization for each hen was a water-in-oil emulsion (50:50) that consisted of 3 mg PLA₂ dissolved in 0.5 mL phosphate buffer saline (PBS), and emulsified with 0.5 mL of Freund Complete Adjuvant (FCA; DIFCO, Sparks, Md.). Seven days after the primary immunization, each hen received a booster injection containing 3 mg PLA₂ dissolved in 0.5 mL PBS and emulsified with Freund Incomplete Adjuvant (FIA; DIFCO). All injections (primary and booster) were administered i.m. into each breast and thigh of hen (1 ml/hen at 0.25 mL/injection site). In all experiments, immunization of hens (primary and booster) occurred at approximately 10 A.M. to eliminate the potential confounding effect of endogenous CORT levels fluctuating due to circadian rhythms.

Based on previous experimentation (Trott et al., 2008; Schade et al., 2001, “Chicken egg yolk antibodies: Production and application,” Springer, Heidelberg, Germany) week three after primary immunization represented peak antibody titer in egg yolk; therefore, eggs were collected once per week beginning on week three and continuing until week eight or ten for repeated testing of anti-PLA₂ antibody content using an ELISA (Trott, et al., 2008).

Anti-PLA₂ antibody content of egg yolk and serum samples were measured by an ELISA (Trott, et al., 2008). A 96-well Nunc-Immuno Plate with MaxiSorp surface (Thermo Fischer Scientific, Waltham, Mass.) was coated overnight (100 μl per well) with PLA₂ (Sigma Aldrich, St. Louis, Mo.) at a concentration of 10 μg/ml in 50 mM sodium bicarbonate. After washing, the plate was blocked (175 μl per well) for at least 1 hour with PBS containing 1% albumin from bovine serum albumin (BSA; Sigma Aldrich). Egg antibody was water-extracted from liquid egg yolk samples. Liquid egg yolk samples (2004) were pipetted with a positive displacement pipette (Rainin, Oakland, Calif.) and extracted overnight with 1.8 ml acidified PBS (pH 5). The extraction mixture was centrifuged at 1,500×g for 10 minutes, and the supernate was further diluted to 1:16,000 with PBS containing 1% BSA (pH 7). Serum samples were diluted 1:16,000 in PBS containing 1% BSA. In addition to the weekly egg yolk or serum samples, an ‘in-lab standard’ was applied to each ELISA plate. The standard applied to each plate consisted of a 2-fold serial dilution from 1:2,000 to 1:64,000 of water-extracted egg yolks from hens immunized against PLA₂ in a previous trial. After coating, blocking, and washing the plate, duplicate samples and ‘in-lab standard’ (100 μl/well) were incubated for 1 hour on the plate followed by washing (6×). The detection antibody, goat anti-chicken IgG-Fc conjugated with horse-radish peroxidase (Bethyl Laboratories, Montgomery, Tex.), was diluted 1:50,000 in PBS and added to the wells (100 μl/well) for 45 minutes followed by washing (8×). Substrate solution (50 mM sodium acetate) containing 0.1 mg/ml tetramethyl benzidine and 3 mM hydrogen peroxide was added (120 per well) for color development (˜15 minutes) and the enzymatic reaction was stopped by addition of 50 μl per well 0.5 M sulfuric acid. Absorbance at 450 nm was measured with a BioTek EL800 plate reader (Winooski, Vt.). Data expressed as Log₂ titer were calculated by comparing samples with the in-lab standard. Titer was defined as the highest dilution of sample with an optical density equal to the standard diluted 1:64,000. Fold-change in titer due to treatment was calculated: [2̂(Log₂ titer of treatment eggs)]/[2̂(Log₂ titer of control eggs)].

Peripheral Blood Mononuclear Cell Counts. In response to feeding CORT (20-80 mg/kg diet), the number of circulating lymphocytes in chicken blood was markedly decreased (Gross et al., 1980, “Some effects of feeding corticosterone to chickens,” Poult. Sci., 59:516-522), and decreased blood lymphocytes correlate with increased serum CORT levels (Gross & Siegel, 1983, “Evaluation of the heterophil/lymphocycte ratio as a measure of stress in chickens,” Avian Dis., 27(4):972-979). Hence, in some experiments peripheral blood mononuclear cell (PBMC) counts were measured to determine physiological effectiveness of dietary CORT administration. Whole blood (2-3 ml) was collected from wing vein into heparin-containing tubes (Becton Dickinson, Sparks, Md.). In 15-ml conical tubes, blood (2 ml) was layered on an equal volume (2 ml) of 1077 Histopaque (Sigma-Aldrich), and centrifuged at 400×g for 30 minutes at room temperature. Mononuclear cells were collected from the gradient interface, and re-suspended in PBS to the original blood volume (2 ml) or the sample. PBMCs were further diluted (1:16) in PBS, mixed with an equal volume of 0.4% trypan blue solution (Sigma-Aldrich), and counted, microscopically, with the aid of a hemacytometer.

Measurement of body weight and egg production. Dietary CORT fed for 1 week at a level of 30 mg/kg diet has been shown to negatively affect body weight gain in broilers (Lin et al., 2006, “Impaired development of broiler chickens by stress mimicked by corticosterone exposure,” Comp. Biochem. Phy., 143(3):400-405), and egg production in laying hens (Wolford et al., 1983, “Reproductive response of laying hens to corticosterone feeding,” Poult. Sci., 62(7):1525). Initial body weight of adult laying hens, 26- to 60-weeks of age, was reported when measured at the beginning of experiments. Data were expressed as a percentage of initial body weight (% initial BW) to determine effects of CORT on change in body weight. Eggs produced were recorded daily for each hen. Laying frequency (or egg production) of each hen during each 7 day period measured, was obtained by dividing the total number of eggs laid by 7.

Statistical Analysis. Data collected from each Example were analyzed by PROC MIX procedure using SAS commercial statistical program (Littell et al, 1996, SAS Systems for Mixed Models, SAS Institute, Inc., NC, USA). For each Example, all data were analyzed by PROC MIX for repeated measures, and probability of treatment difference (P) was reported. Within-week effects of treatment were reported if there was a significant treatment X week interaction effect (p<0.05).

### Example 1

In this Example, hens, 60-weeks of age, were subjected to a short-term moderate feed restriction and antibody response to soluble protein antigen (SPA), as measured by egg yolk antibody, was assessed.

Specifically, control hens (n=20) had ad libitum access to standard corn-soybean meal based laying hen diet. “Feed-restricted” treatment hens (n=20) were given 80 g feed/hen/day for 2 days beginning on day 1 before primary immunization. This level of feed restriction was selected to assure hens would continue to produce eggs. Twenty-four hours after primary immunization, all hens had ad libitum access to standard corn-soybean meal based laying hen diet. Antibody response to SPA was measured from weeks 3 to 9 after primary immunization by ELISA. Body weight was measured before and after two day feed restriction period.

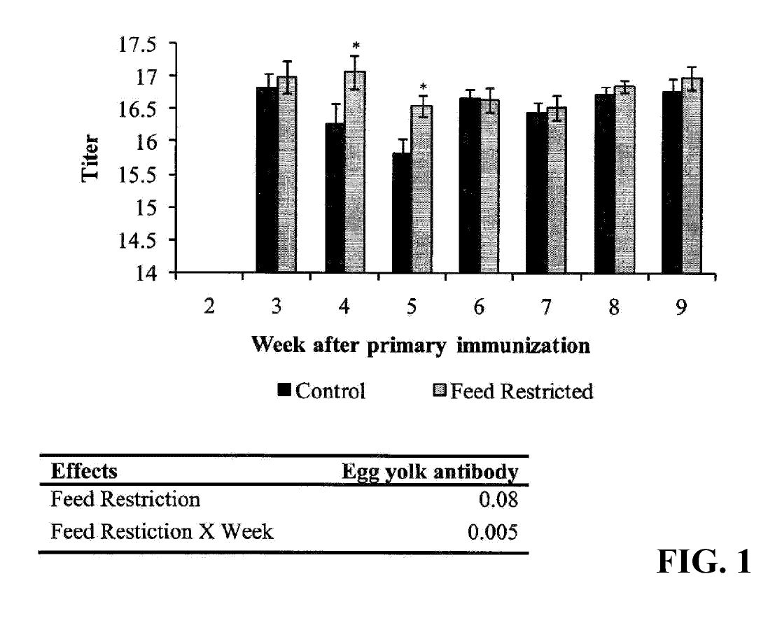

To determine the effect of feed restriction on egg yolk antibody titer, data were analyzed by repeated measures over the entire study period (weeks 3 to 9 after primary immunization). The average antibody titer of egg yolks from feed restricted hens (Log₂ titer=16.80) was 1.2-fold higher than the titer of yolks from control hens (Log₂ titer=16.50; p=0.08; FIG. 1). Data were analyzed within each week because there was an overall interaction effect of feed restriction X week (p=0.005). Antibody titer of egg yolks from feed restricted hens was increased 1.7-fold on week 4 (Log₂ titer=17.06) and week 5 (Log₂ titer=16.55) after primary immunization as compared to the titer of egg yolks from control hens sampled on week 4 (Log₂ titer=16.26; p=0.006) and week 5 (Log₂ titer=15.82; p=0.012) after primary immunization (FIG. 1). At the beginning of the experiment SCWL hens, 60-weeks of age, weighed 1,627±25 g, and there was no effect of feed restriction on change in body weight after two day feed restriction (p=0.22).

### Example 2

In this Example, CORT was fed to hens at the level of either 0 mg/kg diet (n=12) or 30 mg/kg diet (n=12) for 2 days beginning on day 1 before primary immunization. The 2-day duration of administration was selected based on previous research that reported a significant increase in plasma CORT concentrations after 1 day of CORT administered at a level 20 mg/kg diet in drinking water (Post et al., 2003, “Physiological effects of elevated plasma corticosterone concentrations in broiler chickens—An alternative means by which to assess the physiological effects of stress,” Poult. Sci., 82(8):1313-1318). Antibody response to SPA of “control-fed” and “CORT-fed” hens was measured in egg yolks collected from weeks 3 to 6 after primary immunization by ELISA.

During weeks 3 to 6 after primary immunization, there was no difference in antibody titer of egg yolks from control-fed hens (Log₂ titer=15.46) and egg yolks from CORT-fed hens (Log₂ titer=15.52; p=0.24) administered CORT for 2 days beginning on day 1 before primary immunization. Furthermore, there was no treatment X week interaction (p=0.32).

### Example 3

In this Example, CORT was administered to treatment hens similar to Example 2 except that the period of administration was 5 days beginning on day 7 before immunization. The timing and duration of CORT administration was chosen to determine if CORT administered before immunization can indirectly affect antibody response (via leukocyte redistribution). Another reason CORT was administered beginning one week before primary immunization was to minimize the effects of CORT on egg production during peak antibody response (3 weeks after primary immunization; Trott et al., 2008).

Antibody titer of egg yolks from control-fed (n=8) and treatment-fed (n=8) hens collected from week 3 to 5 after primary immunization was measured by ELISA. Body weight (BW) change, PBMC count, and egg production was measured because CORT administered for more than 2 days was expected to decrease body weight (Lin et al., 2006), PBMC count (Gross et al., 1980), and egg production (Wolford et al., 1983). Wolford et al. reported that egg production was completely suppressed in hens after 1 week of dietary CORT (20-40 mg/kg diet), and hens remained out of production for an additional 9-12 days after removal of CORT.

BW and PBMC counts of hens were measured on day 0 of trial when diet administration began. BW were measured on weeks 1, 2, and 5 after start of trial, and PBMC counts were measured on weeks 1 and 2 after start of trial. All hens were immunized on week 1; therefore, week 1 is the only data point measuring the effect of 5-day CORT administration on BW change and PBMC count. Measurements at weeks 2 and 5 could have been influenced by the CORT treatment, the applied immunization, or a CORT X immunization interaction.

During weeks 3 to 5 after primary immunization, there was no difference in antibody titer of egg yolks from control-fed hens (Log₂ titer=17.05) and egg yolks from CORT-fed hens (Log₂ titer=17.02; p=0.96) administered CORT for 5 days beginning on day 7 before primary immunization. There was no treatment X week interaction (p=0.74).

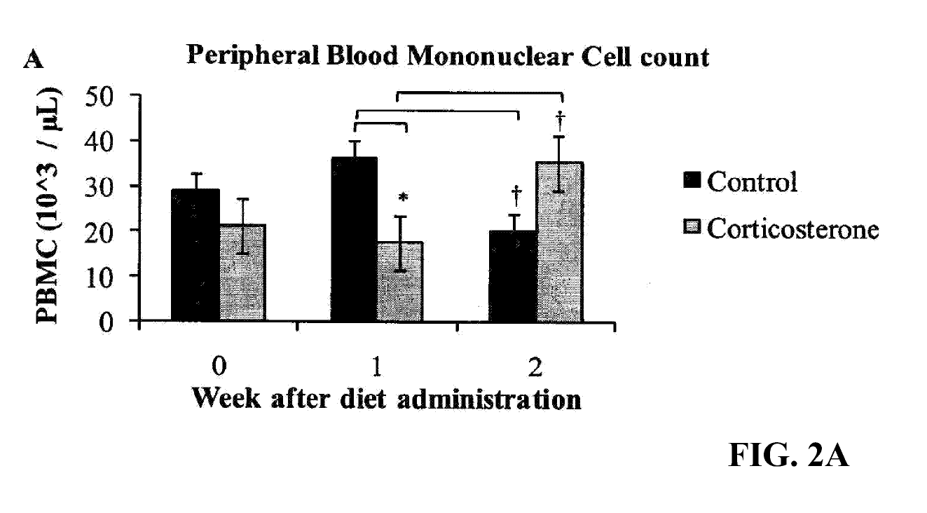

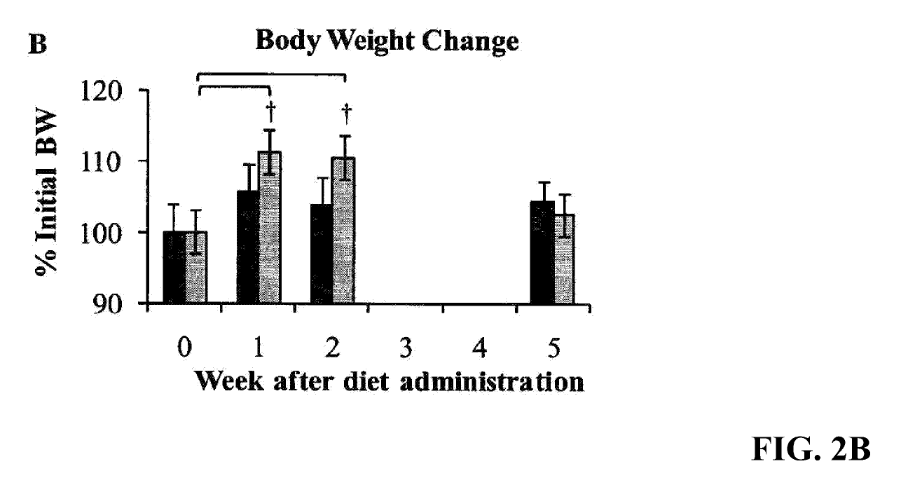

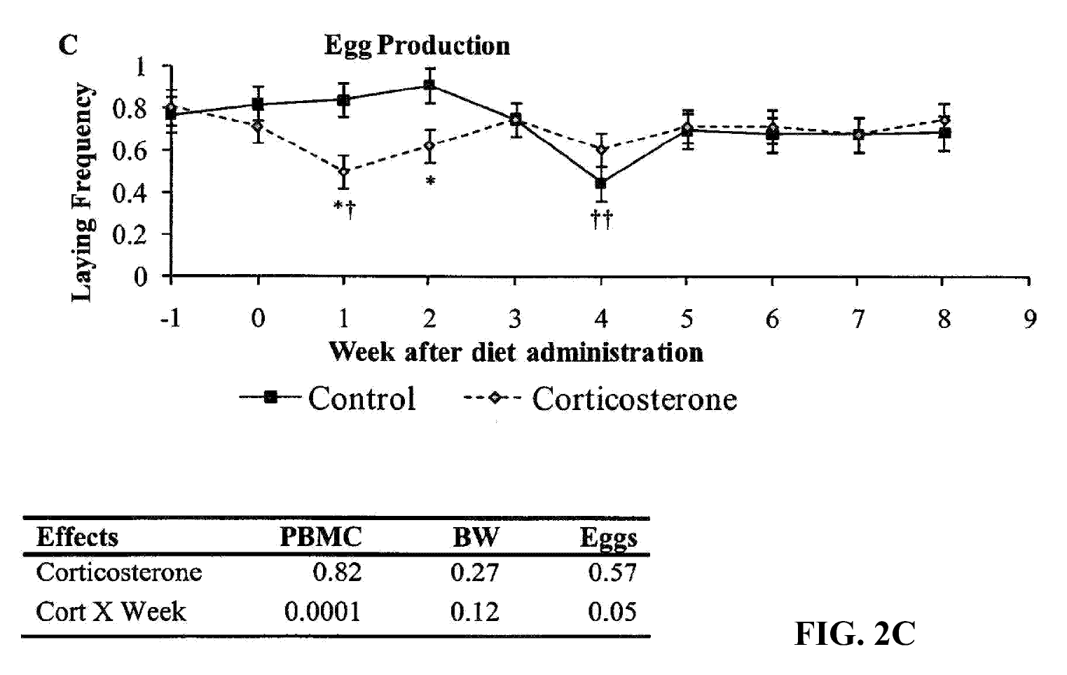

There was no effect of CORT on PBMC counts (p=0.82); however, there was a CORT X week interaction effect (p=0.0001). When PBMC count data were analyzed within each week, the PBMC count of blood sampled from CORT-fed hens was 17,800 cells/μl blood or 49% of the PBMC count of control-fed hens (36,500 cells/μl blood) on week 1 after diet administration (p=0.025; FIG. 2a). Significant differences were detected when data were analyzed between-weeks for control- and CORT-fed hens. PBMC count of control-fed hens was decreased from 36,500 cells/μl blood on week 1 to 20,100 cells/μl blood on week 2 after diet administration (p<0.0001), and PBMC count of CORT-fed hens was increased from 17,800 to 35,400 cells/μl blood (p=0.005) on week 1 and week 2; respectively (FIG. 2a).

As compared to control-fed hens, there was no effect of CORT on body weight (% initial BW; p=0.27), and there was no interaction effect of CORT X week (p=0.12; FIG. 2b). Significant differences were detected when body weight data were analyzed between-weeks for CORT-fed hens. Body weight of CORT-fed hens was 11.4% higher than initial body weight on week 1 after diet administration (p=0.004), and was 10.5% higher than initial body weight on week 2 (p=0.009; FIG. 2b). Body weight of CORT-fed hens was not different from initial body weight on week 5 after diet administration (p=0.57; FIG. 2b).

Throughout this Example, there was no overall difference in egg production of CORT-fed hens as compared to control-fed hens (73% versus 69%; p=0.57); however, there was a significant CORT X week interaction effect (p=0.05; FIG. 2c). Egg production of CORT-fed hens was 34% less than control-fed hens on week 1 after diet administration (50% versus 84%; p=0.005), and 29% less than control-fed hens on week 2 (62% versus 91%; p=0.014; FIG. 2c). Similarly, when data were analyzed between-weeks, egg production of CORT-fed hens was decreased by 21% between week 1 before diet administration (71%) and week 1 after diet administration (50%; p=0.004; FIG. 2c). Between-week analysis of data indicated that egg production of control-fed hens was decreased from 75% to 53% between weeks 3 to 4 (p<0.001) and was increased from 53% back to 70% between weeks 4 to 5 after diet administration (p=0.004; FIG. 2c). The one week decrease in egg production of control-fed hens occurred 3 weeks after primary immunization.

### Example 4

In this Example, the effect of immunization on PBMC counts, BW change, and egg production was determined.

Specifically, thirty-two hens were used in a 2×2 factorial (8 hens per treatment) with two levels of immunization (immunized or nonimmunized) and two levels of CORT administration (0 mg/kg diet or 30 mg/kg diet). The hens receiving CORT (n=16) were administered CORT for 6 days beginning on day 1 before immunization. In contrast to Example 3, CORT was fed before and during primary immunization to insure decreased blood PBMC (Gross et al., 1980) and potentially increase redistribution of lymphocytes to spleen for antigen processing (Mashaly et al., 1993, “The endocrine function of the immune cells in the initiation of humoral immunity,” Poult. Sci., 72:1289-1293). Serum antibody of immunized hens was measured by ELISA at weeks 2 and 3 after immunization because of expected cessation of egg production during this time.

Egg yolk antibody of immunized hens was measured by ELISA at weeks 3 to 9 after primary immunization. BW and PBMC count of hens was measured on day 0 of trial when diet administration began, and again on weeks 1, 2, and 3 after start of trial. Egg production was recorded throughout the trial from weeks 0 to 10.

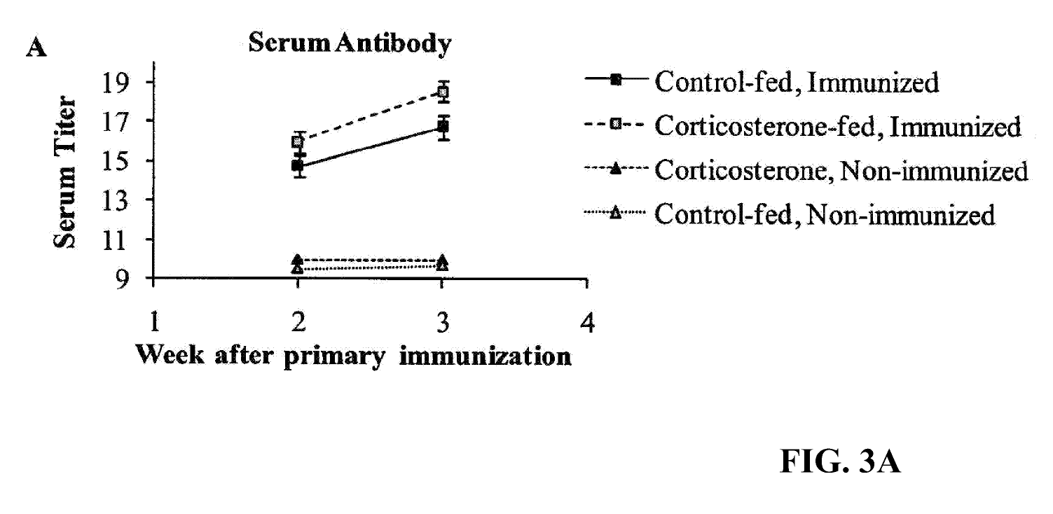

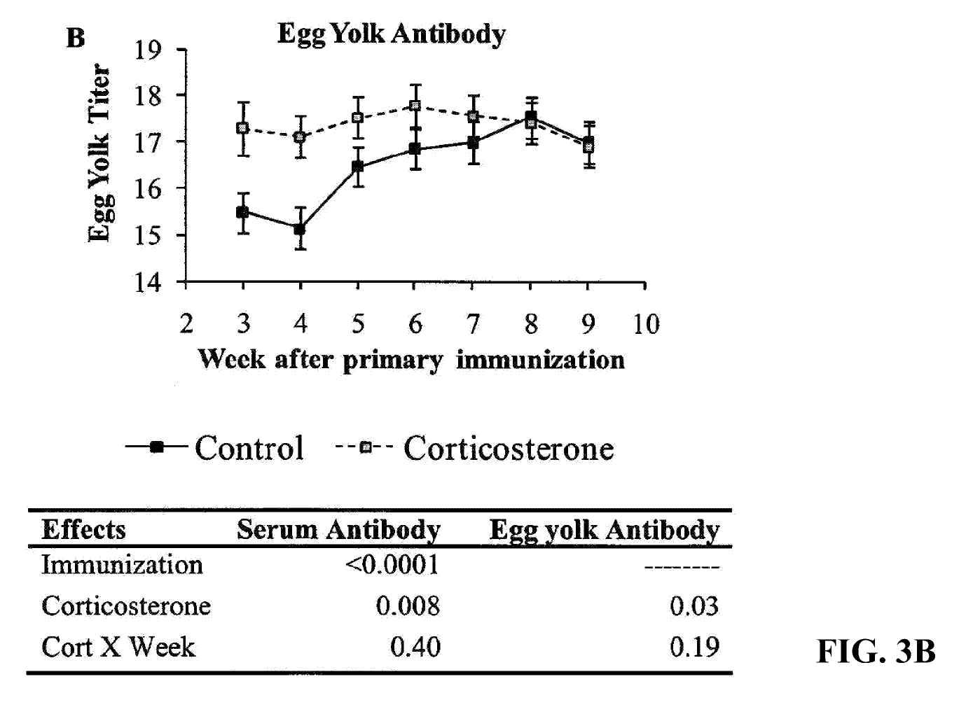

Serum antibody titer of non-immunized hens (Log₂ titer=9.96) was essentially zero as compared to serum antibody titer of immunized hens (Log₂ titer=15.90) on week 2 after primary immunization (p<0.0001). As compared to immunized control-fed hens, serum and egg yolk antibody titers were higher in immunized hens fed CORT for 6 days beginning on day 1 before primary immunization (FIG. 3). Serum antibody titer was 4.9-fold higher in CORT-fed hens (Log₂ titer=17.65) as compared to the serum antibody titer of control hens (Log₂ titer=15.35) from weeks 2 to 3 after primary immunization (p=0.008; FIG. 3a). Corresponding to the serum antibody titer, egg yolk antibody titer was 3.1-fold higher in eggs from CORT-fed hens (Log₂ titer=17.32) as compared to eggs from control-fed hens (Log₂ titer=15.69) collected from weeks 3 to 5 after primary immunization (p=0.006). Over the entire study period (weeks 3 to 9), there was a 1.8-fold increase in antibody titer of egg yolks from CORT-fed hens (Log₂ titer=17.37) as compared to the titer of egg yolks from control hens (Log₂ titer=16.49; p=0.032; FIG. 3b). There were no CORT X week interaction effects.

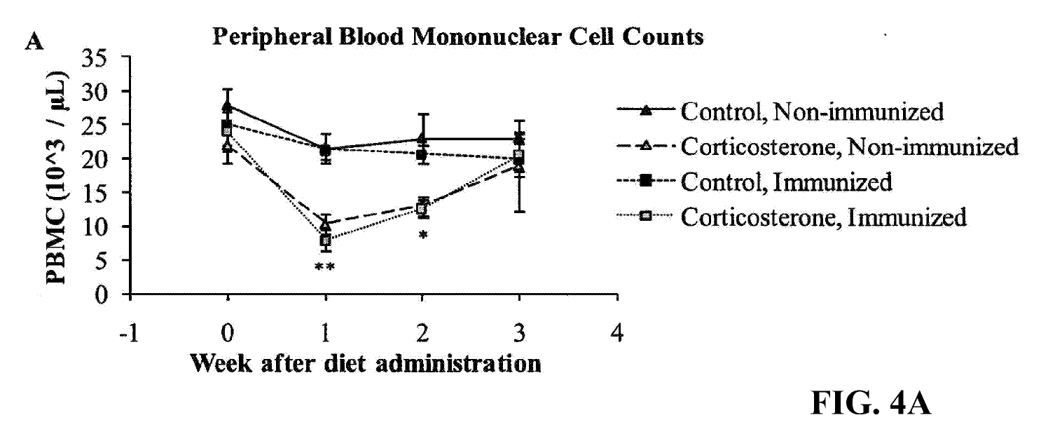

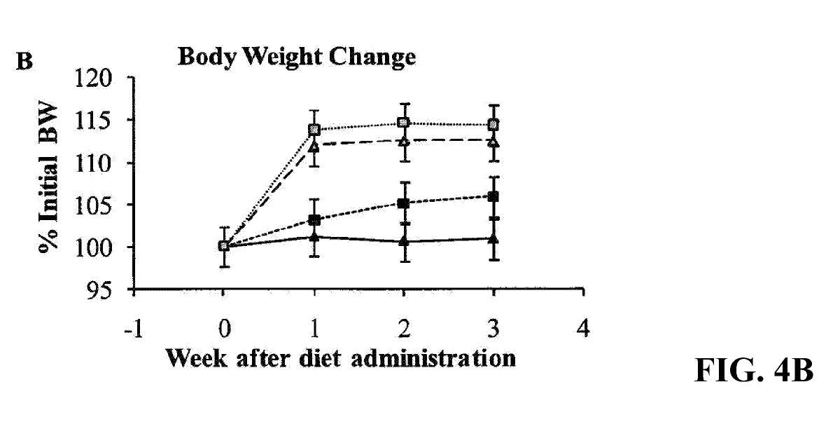

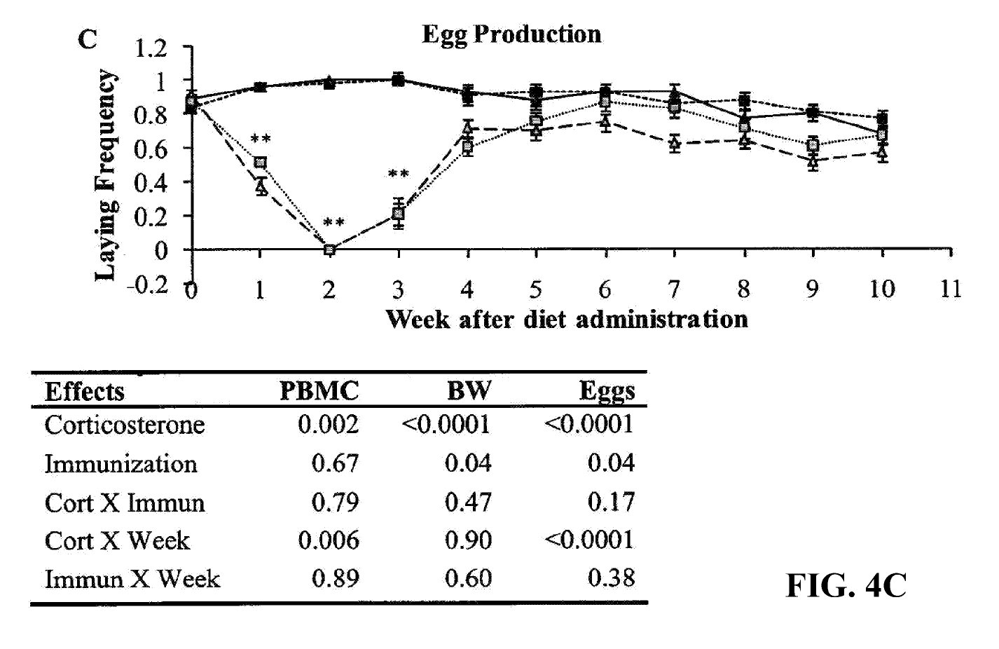

Changes in blood PBMC counts were due to the main effect of dietary CORT (p=0.002; FIG. 4a). The effects of immunization (p=0.67), CORT X immunization (0.79), and immunization X week (0.89) were not significant. There was a significant CORT X week interaction effect (p=0.006); hence, data were analyzed within-week. PBMC count of CORT-fed hens (9,200 cells/μl blood) was 43% of the PBMC count of control-fed hens (21,400 cells/μl blood) on week 1 after diet administration (p<0.0001), and 59% on week 2 (12,900 versus 21,900 cells/μl blood; p=0.001; FIG. 4a). PBMC counts returned to control levels on week 3 after primary immunization (19,700 versus 21,300 cells/μl blood; p=0.57; FIG. 4a).

Changes in body weight were due to the main effect of dietary CORT (p<0.0001) and the main effect of immunization (p=0.04; FIG. 4b). There were no interaction effects on body weight. Body weight of CORT-fed hens was 13.3% higher than initial body weight on weeks 1 to 3 after diet administration (FIG. 4b). Body weight of immunized hens was 9.5% higher than initial body on weeks 1 to 3 after immunization (FIG. 4b).

The main effects of CORT (p<0.0001) and immunization (p=0.04) on egg production were detected. Overall egg production of CORT-fed hens (54%) was 35% less than egg production of control-fed hens (89%), and overall egg production of immunized hens (74%) was 4% higher than non-immunized hens (70%; FIG. 4c). A significant CORT X week interaction effect on egg production was detected (p<0.0001). As compared to control-fed hens, egg production of CORT-fed hens was suppressed 50%, 100%, and 80% on weeks 1, 2, 3 after diet administration; respectively (p<0.0001; FIG. 4c). Thereafter, egg production of CORT-fed hens (69%) was 17% lower than egg production of control-fed hens (86%) from weeks 4 to 10 after primary immunization (p<0.0001; FIG. 4c). No other significant effects were detected.

### Example 5

In this Example, CORT was administered for 9 days beginning on day 1 before primary immunization and ending on day 8 after primary immunization.

After stimulating initiation of antibody response, CORT may inhibit antibody response in a negative feedback mechanism (Mashaly et al., 1998, “The role of neuroendocrine immune interactions in the initiation of humoral immunity in chickens,” Dom. Anim. Endocrinology, 15(5):409-422); hence, in contrast to previous Examples, it was hypothesized that CORT administered for a longer duration (9 days) may inhibit antibody response. Additionally, any effects on antibody response in this Example were more likely to be directly attributed to exogenous CORT because hens were exposed to dietary CORT during both the primary (day 0) and booster immunization (day 7).

Hens were given dietary CORT throughout both the primary immunization (day 0) and booster immunization (day 7) to determine effects of CORT on egg yolk antibody specific to SPA and on egg production.

Antibody response to SPA of hens was measured in egg yolks collected from weeks 3 to 6 after primary immunization by ELISA. Egg production was recorded throughout the experiment from week 0 to week 10.

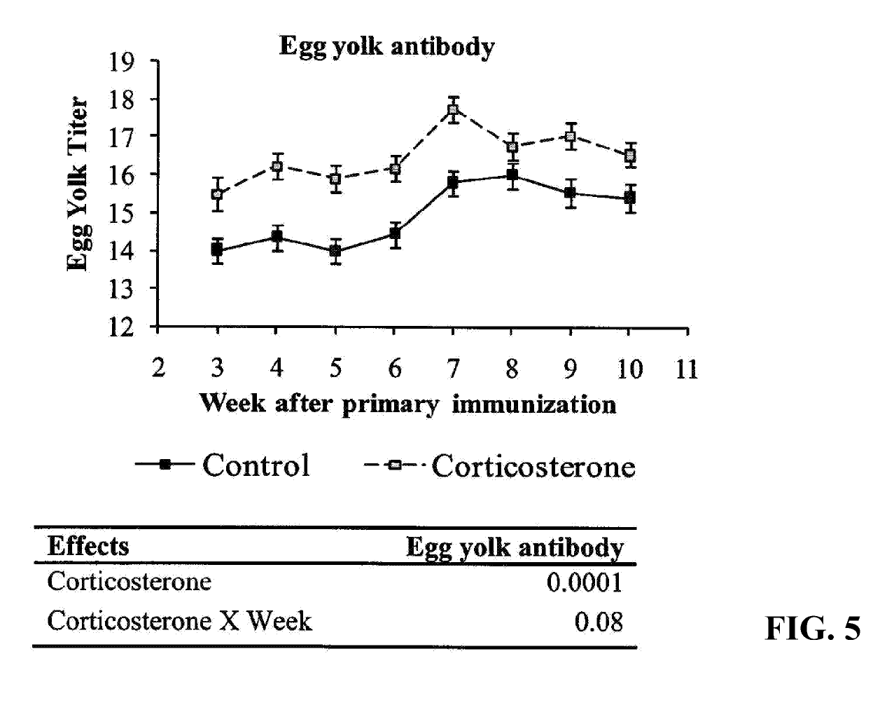

When CORT was fed to treatment hens for 9 days beginning on day 1 before primary immunization, antibody titer of egg yolks from CORT-fed hens (Log₂ titer=16.50) was 3.0-fold higher than egg yolks from control-fed hens (Log₂ titer=14.95) collected from weeks 3 to 10 after primary immunization (p=0.00001; FIG. 5). There was not a significant CORT X week interaction effect (p=0.08).

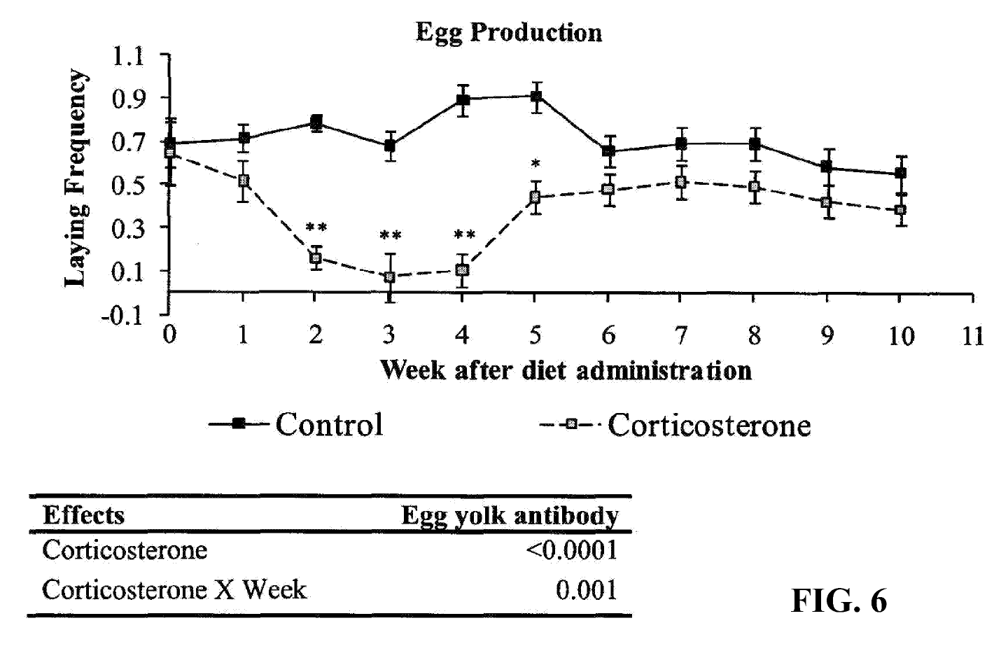

A main effect of CORT (p<0.0001) on egg production was detected. Overall egg production of CORT-fed hens (39%) was 31% less than egg production of control-fed hens (70%; FIG. 6). A significant CORT X week interaction effect on egg production was detected (p=0.001). As compared to control-fed hens, egg production of CORT-fed hens was suppressed 60% on weeks 2 and 3, and 80% on week 4 after diet administration (p<0.0001; FIG. 6). On week 5 after diet administration, egg production was 50% lower in CORT-fed hens (45%) as compared to control-fed hens (91%; p=0.007); thereafter, egg production of CORT-fed hens (53%) was 14% lower than egg production of control-fed hens (67%) from weeks 6 to 11 after primary immunization (p=0.001; FIG. 6).

We claim:

1. A method for making an egg having a yolk that comprises an antigen-specific antibody, the method comprising the steps of:
administering a corticosteroid to an egg-laying animal; and
exposing the animal to an immunogenic dose of an antigen.

2. The method as set forth in claim 1, wherein the antigen is administered at least about 30 minutes after administration of the corticosteroid.

3. The method as set forth in claim 1, wherein the antigen is administered at least 24 hours after administration of the corticosteroid.

4. The method as set forth in claim 1, wherein the antigen is administered at least 48 hours after administration of the corticosteroid.

5. The method as set forth in claim 1, wherein the corticosteroid is administered to the animal for a period of at least 1 day.

6. The method as set forth in claim 1, wherein the corticosteroid is administered to the animal for a period of at least 2 days.

7. The method as set forth in claim 1, wherein the corticosteroid is administered to the animal for a period of at least 5 days.

8. The method as set forth in claim 1, wherein the corticosteroid is administered to the animal for a period of at least 6 days.

9. The method as set forth in claim 1, wherein the animal is selected from the group consisting of an avian, a mammal, a marsupial, a reptile, and an amphibian.

10. The method as set forth in claim 9, wherein the avian animal is selected from the group consisting of a chicken, quail, pheasant, a duck, an emu, a goose, an ostrich, and a turkey.

11. A method as set forth in claim 10, wherein the avian animal is a chicken.

12. A method as set forth in claim 1, wherein the corticosteroid is selected from the group consisting of corticosterone, dexamethasone, and cortisol.

13. A method as set forth in claim 1, wherein the corticosteroid is cortisol.

14. An egg having an antigen-specific egg yolk antibody titer of at least 0.28-0.5 mg/ml, wherein the egg is produced according to a method comprising the steps of:
administering a corticosteroid to an egg-laying animal; and
exposing the animal to an immunogenic dose of the antigen, the corticosteroid being administered in an amount of sufficient to increase titer of the antigen-specific antibody relative to the titer of antibody specific to the antigen in an animal exposed to the immunogenic dose without the corticosteroid.

15. The egg as set forth in claim 14, wherein the antigen-specific egg yolk antibody titer is at least 2.0-fold higher than that of an egg produced without administering the corticosteroid to the animal.

16. The egg as set forth in claim 14, wherein the antigen-specific egg yolk antibody titer is at least 2.5-fold higher than that of an egg produced without administering the corticosteroid to the animal.

17. The egg as set forth in claim 14, wherein the antigen-specific egg yolk antibody titer is at least 3.0-fold higher than that of an egg produced without administering the corticosteroid to the animal.

18. The egg as set forth in claim 15, wherein the egg is an avian egg.

19. The egg as set forth in claim 18, wherein the avian egg is selected from the group consisting of a chicken egg, quail egg, pheasant egg, a duck egg, an emu egg, a goose egg, an ostrich egg and a turkey egg.

20. The egg as set forth in claim 19, wherein the avian is a chicken.
